(19) United States
(12) Patent Application Publication
(10) Pub. No.: US 2024/0226067 A9
(43) Pub. Date: Jul. 11, 2024

(54) ANTI-FIBROTIC THERAPIES

(71) Applicant: THE JOHNS HOPKINS UNIVERSITY, Baltimore, MD (US)

(72) Inventors: Florin M. Selaru, Baltimore, MD (US); Laura Ensign-Hodges, Baltimore, MD (US); Ling Li, Baltimore, MD (US); Min Kyung Joo, Baltimore, MD (US)

(21) Appl. No.: 18/548,423

(22) PCT Filed: Mar. 1, 2022
(86) PCT No.: PCT/US2022/018398

§ 371 Date: Aug. 30, 2023

Related U.S. Application Data
(60) Provisional application No. 63/155,609, filed on Mar. 2, 2021.

(51) Int. Cl. A61K 31/4174; A61P 1/00

(52) U.S. Cl. CPC A61K 31/4174; A61P 1/00

(57) ABSTRACT

The present disclosure provides methods, uses, and pharmaceutical compositions comprising and azole derivative (e.g., sulconazole) in the treatment or prevention of fibrotic disease or disorder or fibrosis, including Crohn's disease.

### CROSS-REFERENCE TO RELATED APPLICATIONS

This application claims the benefit of U.S. Provisional Application No. 63/155,609, filed Mar. 2, 2020, the content of which is herein incorporated by reference in its entirety.

### STATEMENT REGARDING FEDERALLY SPONSORED RESEARCH OR DEVELOPMENT

This invention was made with government support under DK107806 awarded by the National Institutes of Health. The government has certain rights in the invention.

### TECHNICAL FIELD

The present disclosure relates to methods and compositions for treating a subject having a fibrotic disease or disorder or at risk of developing fibrosis.

### BACKGROUND OF THE INVENTION

The progressive accumulation of fibrous tissue is thought to represent a relative imbalance between pro-fibrotic processes, which lay down connective tissue, and anti-fibrotic processes, which cause resorption of connective tissue. The net result of the imbalance is the replacement of normal cells by dense fibrous bands of protein, indicative of fibrosis or a fibrotic disease or disorder.

Within 20 years of diagnosis, up to 20% of Crohn's disease (CD) patients will develop fibrosis, resulting in intestinal strictures. Endoscopic dilation is possible only in select cases, the majority require surgery which can result in loss of bowel, morbidity, and mortality. Even with interventions, intestinal strictures almost invariably recur, resulting in further surgery, abdominal adhesions, loss of bowel, short gut, and/or other complications. Thus, one of the major unmet clinical needs in the management of inflammatory bowel diseases (IBD) is the management of Crohn's patients with intestinal strictures.

Historically, the prevailing view was that fibrosis in Crohn's disease patients was due to inflammation alone, and therefore, could be effectively treated or prevented by treating the inflammation. However, the past 20 years of clinical experience treating Crohn's patients with potent anti-inflammatory biologics (anti-TNF-α, anti-IL-12/23, etc.) has revealed little to no impact on the progression of fibrostenosing complications in Crohn's patients. The end result is that Crohn's patients with intestinal fibrosis continue to necessitate surgical resection of the diseased, fibrosed segment of the gut. However, there are currently no FDA-approved therapies for treating intestinal fibrosis or preventing the recurrence of intestinal strictures that are a result of fibrosis. Some early efforts with localized injections of anti-inflammatory agents, such as triamcinolone or anti-fibrotic nucleic acids directly into the inflamed tissue, have not produced clinically meaningful results. Indeed, a single injection of a drug or nucleic acids in solution will have a very short duration of action before being cleared from the body, whereas tissue remodeling processes like fibrosis occur over days and weeks.

### SUMMARY OF THE INVENTION

The present invention is directed to methods and compositions for treating a subject having a fibrotic disease or disorder or at risk of developing fibrosis. The methods may comprises administering an effective amount of sulconazole or a pharmaceutically acceptable salt thereof.

A method of treating a subject having a fibrotic disease or disorder or at risk of developing fibrosis, the method comprising administering an effective amount of an azole derivative (e.g., sulconazole), or a pharmaceutically acceptable salt thereof, to the subject. In some embodiments, the fibrotic disease or disorder comprises Crohn's disease (CD). In some embodiments, the subject has or has a history of intestinal strictures. In some embodiments, the azole derivative, or a pharmaceutically acceptable salt thereof, is administered systemically or locally to the site of the fibrosis or the fibrotic disease or disorder.

The present invention is also directed to a pharmaceutical composition for the treatment of a subject having a fibrotic disease or disorder or at risk of developing fibrosis comprising: an effective amount of an azole derivative (e.g., sulconazole), or a pharmaceutically acceptable salt thereof; and a pharmaceutically acceptable carrier. In some embodiments, the fibrotic disease or disorder comprises Crohn's disease (CD).

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is images of cells from high throughput microscopy-based analysis of an FDA-approved drug library for potential anti-fibrotic properties. Cells are CCO18CD colon fibroblasts stimulated with TGF-β to induce fibrotic processes demonstrated by increased production of alpha smooth muscle actin (α-SMA, red color) and intracytoplasmic collagen 1 (green color), as seen when no anti-fibrotic drug treatment was given (Untreated). Sulconazole caused similar reductions in α-SMA and collagen production at much lower concentrations than the pulmonary anti-fibrotic drug, pirfenidone (4 mM versus 10 μM, a 400-fold lower concentration).

FIG. 2 is graphs of the expression of key fibrosis genes (α-SMA (ACTA2), collagen type 1 (COL1A1), collagen type 3 (COL3A1), TGF-β receptor 1 (TGFBR1)) in transplanted intestinal tissue after various treatments: single intraperitoneal injection of phosphate buffered saline (PBS, n=11), three times daily oral gavage administration of pirfenidone (Pirf Oral 3× Daily, 100 mg/kg, n=5), and one subcutaneous injection of free sulconazole (Free Sulc SubQ, 15 mg/kg, n=2), # indicates only n=2 mice in Free Sulc SubQ treatment due to death of mice at 50 mg/kg dose. Data normalized to PBS and shown as mean±SEM, *p<0.05 compared to PBS control.

FIGS. 3A-3C are images of cells from high throughput microscopy-based analysis of potential drugs for anti-fibrotic properties. Cells are CCO18CD colon fibroblasts stimulated with TGF-β (FIG. 3B) to induce fibrotic processes demonstrated by increased production of alpha smooth muscle actin (α-SMA, red color) and intracytoplasmic collagen I (green color).

### DETAILED DESCRIPTION OF THE INVENTION

The present disclosure provides methods, uses, and pharmaceutical compositions for use in the treatment or prevention of a fibrotic disease or disorder or fibrosis.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, small molecule chemistry and animal models described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing,” and “introducing” are used interchangeably herein and refer to the placement of a compound (e.g., sulconazole) or compositions thereof into a subject by a method or route which results in at least partial localization a desired site. The compound or compositions can be administered by any appropriate route which results in delivery to a desired location in the subject.

An “azole” and “azole derivative” are used interchangeably herein and refer to compound having a five-membered ring containing a nitrogen atom and at least one other non-carbon atom (e.g., nitrogen, sulfur, or oxygen) as part of the ring (e.g., imidazole, pyrazole, triazoles, and the like). As used herein, “triazole” refers to a compound comprising a five-membered ring having the molecular formula C₂H₃N₃, including 1,2,3-triazole and 1,2,4, triazole. Azoles and azole derivatives may include compounds comprising more than one five-membered ring as described above.

A “fibrotic condition,” “fibrotic disease,” and “fibrotic disorder” are used interchangeably herein to refer to a condition, disease, or disorder that is amenable, at least partially, to treatment by administration of a compound having anti-fibrotic activity.

As used herein, the term “preventing” refers to partially or completely delaying onset of a disease, disorder and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition.

As used herein, “treat,” “treating,” and the like means a slowing, stopping, or reversing of progression of a disease or disorder. The term also means a reversing of the progression of such a disease or disorder. As such, “treating” means an application or administration of the methods or compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human. In some embodiments, the subject is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. TREAT OR PREVENT FIBROTIC DISEASE OR FIBROSIS

The present disclosure provides methods for treating a subject having a fibrotic disease or disorder or at risk of developing fibrosis comprising administering an effective amount of an azole or azole derivative, or a pharmaceutically acceptable salt or composition thereof, to the subject.

Azoles and azole derivatives include, but are not limited derivatives of imidazole (e.g., 1H-imidazole, 2-methyl-1H-imidazole, 1,2-dimethylimidazole, benzimidazole, 2-methylbenzimidazole, 2-phenylimidazole, 4,5-diphenylimidazole, and 2,4,5-triphenylimidazole, 1-[β-(R-Thio)phenethyl]-imidazoles, 1-(β-aryl)ethyl-imidazole, bifonazole, butoconazole, chlormidazole, climbazole, clotrimazole, croconazole, eberconazole, econazole, fenticonazole, flutrimazole, isoconazole, ketoconazole, luliconazole, miconazole, neticonazole, omoconazole, oxiconazole, sertaconazole, sulconazole, tioconazole) and derivatives of triazole (e.g. benzotriazole, triazolopyrimidine, efinaconazole, fluconazole, terconazole, fluconazole, hexaconazole, fosfluconazole, fosravuconazole, isavuconazonium, itraconazole, posaconazole, voriconazole). In some embodiments, the azole derivative comprises one or more of: sulconazole, miconazole, econazole, tioconazole, butoconazole, aliconazole and/or a pharmaceutically acceptable salt thereof.

The methods and compositions herein may comprise sulconazole, or a pharmaceutically acceptable salt thereof. As used herein, the term “sulconazole” refers to the compound having the IUPAC name: 1-[2-[(4-chlorophenyl)methylsulfanyl]-2-(2,4-dichlorophenyl)ethyl]imidazole. The compound is disclosed in U.S. Pat. No. 4,055,652, incorporated herein by reference. In some embodiments, another 1-[β-(R-thio)phenethyl]-imidazole as disclosed in U.S. Pat. No. 4,055,652, may be included in addition to or as a substitution for sulconazole.

The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, nitrate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. In some embodiments, the pharmaceutically acceptable salt of sulconazole is sulconazole nitrate.

Fibrosis is generally characterized by the pathologic or excessive accumulation of collagenous connective tissue. Fibrotic disease and disorders include, but are not limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, and the like), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport's syndrome, HIV-associated nephropathy, polycystic kidney disease, Fabry's disease, diabetic nephropathy, chronic glomerulonephritis, nephritis associated with systemic lupus, and the like), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's opthalmopathy, diabetic retinopathy, glaucoma, Peyronie's disease, penis fibrosis, urethrostenosis after the test using a cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, radiation (e.g., cancer radiotherapy), and the like), and the like.

In some embodiments, the fibrotic disease or disorder comprises an inflammatory bowel disease (IBD). In some embodiments, the fibrotic disease or disorder comprises Crohn's disease (CD). In some embodiments, the subject has or has a history of intestinal strictures. Administration can be initiated as soon as it is determined that the subject is at risk of developing fibrosis or, in the case of Crohn's disease, at risk of developing intestinal strictures.

A wide range of second therapies may be used in conjunction with the methods and compositions described herein. Such second therapies include, but are not limited to, administration of a different therapeutic agent (e.g., anti-inflammatory agents) or surgical or mechanical intervention.

In some embodiments, the methods further comprise performing a surgical intervention or endoscopic dilation. The surgical intervention may comprise surgical resection of the diseased, fibrosed tissue (e.g., intestinal tract). In some embodiments, the administration of a second therapy may be before, after, or substantially simultaneously to the administration of sulconazole. For, example, if a surgical intervention or endoscopic dilation, the azole derivative (e.g., sulconazole) may be administered during and/or after the procedure. In some embodiments, the azole derivative (e.g., sulconazole) is administered at the same time as the surgical intervention or endoscopic dilation. In some embodiments, the azole derivative (e.g., sulconazole) is administered after a period of time following surgical intervention or endoscopic dilation. The period of time may vary, but may be on the time period of days, weeks, or months.

The specific dose level may depend upon a variety of factors including the age, body weight, and general health of the subject, time of administration, and route of administration. An “effective amount” is an amount that is delivered to a subject, either in a single dose or as part of a series, which achieves a medically desirable effect. For prophylaxis purposes, the amount of sulconazole in each dose is an amount which induces a protective result without significant adverse side effects. For treatment purposes, the amount of sulconazole in each dose is an amount which slows or reverses the progression of the disease or at least a subset of the symptoms. The determination of effective dosage levels can be accomplished by one skilled in the art using routine methods, for example, human clinical trials, in vivo studies, and in vitro studies. For example, useful dosages of a compound can be determined by comparing the in vivo activity in animal models.

In some embodiments, the effective amount of the azole derivative (e.g., sulconazole), or a pharmaceutically acceptable salt thereof, is 0.1-50 mg/kg. The effective amount may be greater than 0.1 mg/kg, 0.5 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10 mg/kg, 15 mg/kg, 20 mg/kg, 25 mg/kg, 30 mg/kg, 35 mg/kg, or 45 mg/kg. The effective amount may be less than 50 mg/kg, 45 mg/kg, 40 mg/kg, 35 mg/kg, 30 mg/kg, 25 mg/kg, 20 mg/kg, 15 mg/kg, 10.0 mg/kg, 9.0 mg/kg, 8.0 mg/kg, 7.0 mg/kg, 6.0 mg/kg, 5.0 mg/kg, 4.0 mg/kg, 3.0 mg/kg, 2.0 mg/kg, 1.0 mg/kg, or 0.5 mg/kg. In certain embodiments, the effective amount of sulconazole is 0.1-20 mg/kg. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The frequency of dosing the effective amount can vary, but typically the effective amount is delivered daily, either as a single dose, multiple doses throughout the day, or depending on the dosage form, dosed continuously for part or all of the treatment period.

Administration may be by various routes known to those skilled in the art, including without limitation oral, intravenous, intramuscular, topical, subcutaneous, and/or systemic. In some embodiments the administration may be parenteral administration (including, but not limited to, subcutaneous, intramuscular, intravenous, and intraperitoneal injections). In some embodiments, the azole derivative (e.g., sulconazole) is administered systemically. In some embodiments, the azole derivative (e.g., sulconazole) is administered by injection. In some embodiments, the azole derivative (e.g., sulconazole) is administered locally to the site of the fibrosis or the fibrotic disease or disorder.

The azole derivative (e.g., sulconazole) may be administered in a pharmaceutical composition which further comprises pharmaceutically acceptable carriers. The choice of excipients or pharmaceutically acceptable carriers will depend on factors including, but not limited to, the particular mode of administration, the effect of the excipient on the solubility and stability of the azole derivative, or the pharmaceutically acceptable salt thereof, and the nature of the dosage form.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringers solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Typically, the route by which the azole derivative is administered and the form of any composition thereof will dictate the type of carrier to be used. The use of such pharmaceutically acceptable carriers for pharmaceutically active substances is well known in the art. The compositions and methods for their preparation will be readily apparent to those skilled in the art. Techniques and formulations may be found, for example, in Remington's Pharmaceutical Sciences, 19th Edition (Mack Publishing Company, 1995).

### 3. EXAMPLES

### Example 1

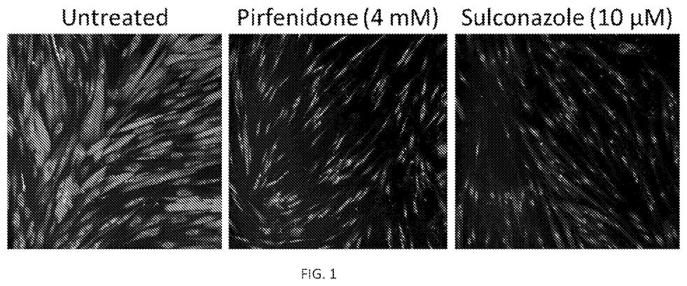

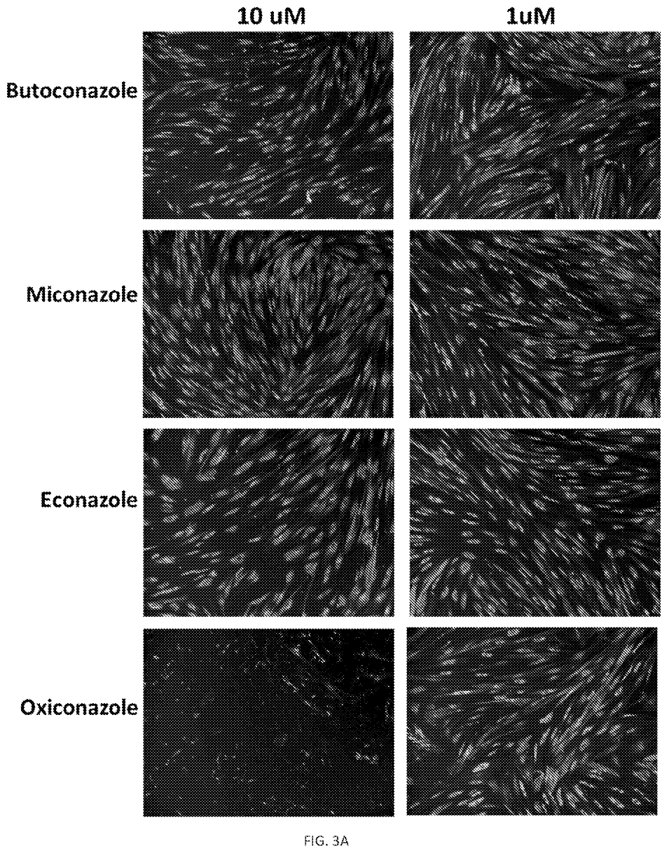

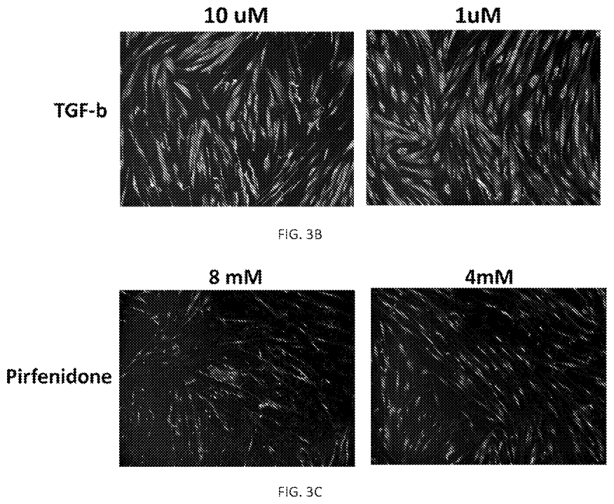

A high throughput microscopy-based drug screening approach using intestinal fibroblast cells was used to screen a library of FDA-approved drug candidates for potential anti-fibrotic activity (FIGS. 1 and 3A-3C). As shown in FIG. 1 and FIG. 3C, pirfenidone, a drug recently approved for treating pulmonary fibrosis, reduced the induced expression of alpha-smooth muscle actin (α-SMA, red) and collagen (green) at millimolar concentrations. In contrast, sulconazole had a similar effect at much lower concentrations, 10 μM (400-fold lower concentration compared to pirfenidone). Consequently, sulconazole was much more potent while also having minimal impact on cell survival at effective concentrations. Other azole derivatives also showed reduction of the induced expression of alpha-smooth muscle actin and/or collagen at sub-millimolar concentrations (FIG. 3A).

### Example 2

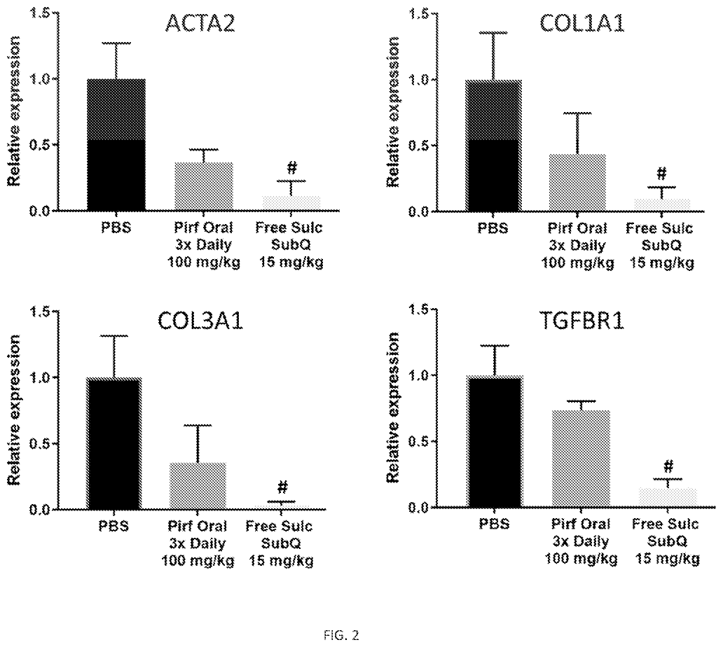

To test the anti-fibrotic properties of sulconazole, a mouse model that was previously used to validate the anti-fibrotic efficacy of oral pirfenidone was used. A 1-2 cm segment of bowel was excised from one mouse and implanted in the subcutaneous space on the neck of another mouse of the same genetic background. The resulting ischemia promotes a strong pro-fibrogenic milieu that resulted in myofibroblast invasion and extracellular collagen deposition. Using this model allowed injection of free sulconazole in the subcutaneous space to mimic local delivery to the small bowel in patients. As a positive control, pirfenidone was utilized at the dose previously shown to be effective (100 mg/kg three times orally per day). Subcutaneous free sulconazole was used at a dose of 15 mg/kg. One week after tissue implantation and initiation of treatment, the segment of transplanted intestine tissue was excised to assess expression of key genes involved in fibrosis (α-SMA, ACTA2; collagen type 1, COL1A1; collagen type 3, COL3A1; TGF-β receptor 1, TGFBR1) by qPCR. As shown in FIG. 2, sulconazole was significantly more effective than pirfenidone at repressing fibrosis genes.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A method of treating a subject having a fibrotic disease or disorder or at risk of developing fibrosis, the method comprising administering an effective amount of an azole derivative, or a pharmaceutically acceptable salt thereof, to the subject.

2. The method of claim 1, wherein the azole derivative is sulconazole, or a pharmaceutically acceptable salt thereof.

3. The method of claim 1 or claim 2, wherein the fibrotic disease or disorder comprises Crohn's disease (CD).

4. The method of claim 3, wherein the subject has a history of intestinal strictures.

5. The method of claim 3 or claim 4, further comprising performing a surgical intervention or endoscopic dilation.

6. The method of claim 5, wherein the azole derivative, or a pharmaceutically acceptable salt thereof, is administered at the same time as the surgical intervention or endoscopic dilation.

7. The method of claim 6, wherein the azole derivative, or a pharmaceutically acceptable salt thereof, is administered after a period of time following the surgical intervention or endoscopic dilation.

8. The method of any of claims 1-7, wherein the azole derivative, or a pharmaceutically acceptable salt thereof, is administered systemically or locally to the site of the fibrosis or the fibrotic disease or disorder.

9. The method of any of claims 1-8, wherein the effective amount of the azole derivative, or a pharmaceutically acceptable salt thereof, is 0.1-50 mg/kg.

10. A pharmaceutical composition for the treatment of a subject having a fibrotic disease or disorder or at risk of developing fibrosis comprising:
an effective amount of an azole derivative, or a pharmaceutically acceptable salt thereof; and
a pharmaceutically acceptable carrier.

11. The pharmaceutical composition of claim 10, wherein the azole derivative is sulconazole.

12. The composition of claim 10 or claim 11, wherein the fibrotic disease or disorder comprises Crohn's disease (CD).

13. Use of an azole derivative, or a pharmaceutically acceptable salt thereof, in the manufacture of a medicament for the treatment or prevention of a fibrotic disease or disorder or fibrosis.

14. The use of claim 13, wherein the azole derivative is sulconazole.

15. The use of claim 13 or claim 14, wherein the fibrotic disease or disorder comprises Crohn's disease (CD).
